(19) United States
(12) Patent Application Publication
(10) Pub. No.: US 2014/0367259 A1
(43) Pub. Date: Dec. 18, 2014

(54) METHOD FOR IDENTIFYING A TARGET POLYMER

(71) Applicant: BASE4 INNOVATION LTD, Cambridge, Cambrigeshire (GB)

(72) Inventors: Cameron Alexander Frayling, Cambridge (GB); Bruno Flavio Nogueira de Sousa Soares, Cambridge (GB); Barnaby Balmforth, Cambridge (GB)

(21) Appl. No.: 14/366,175

(22) PCT Filed: Dec. 20, 2012
(86) PCT No.: PCT/GB2012/053216

§ 371 Date: Jun. 17, 2014

(30) Foreign Application Priority Data
Dec. 20, 2011 (GB) 1122020.9

(51) Int. Cl. G01N 33/487; C12Q 1/68; G01J 3/44; G01N 27/447; G01N 21/65

(52) U.S. Cl. CPC G01N 33/48721; G01N 27/447; G01N 21/658; G01J 3/4406; C12Q 1/6869; C12Q 1/689; G01N 2201/069
USPC 204/452

(57) ABSTRACT

A method for identifying a target polymer (20) comprises translocating a target polymer (20) having detectable elements (22), such as fluorophores (22), through an analysing device (24) comprising a nanopore (28) having a detection window (40), wherein the analysing device (24) is capable of plasmon resonance to produce a localised electromagnetic field which defines the detection window (40) detecting the detectable elements (22) as they pass through the detection window (40) to produce a distribution profile of the detectable elements (22) along the target polymer (20) and identifying the target polymer (20) by comparing the distribution profile to a reference set of distribution profiles for known polymers. In a preferred embodiment the target polymer (20) is a nucleic acid and the detectable elements (22) are oligonucleotides complimentary to at least two adjacent nucleotides therein. Exemplified is the use of 6-mer oligonucleotides.

The present invention relates to a method and apparatus for identifying a target polymer such as a nucleic acid which, for example, is useful in the detection of pathogens.

The polymerase chain reaction (PCR) is used for multiple pathogen detection. PCR is, however, not sufficiently rapid to be adapted to a point-of-care diagnostic tool. Microarrays allow a certain amount of flexibility, but the cost has proved difficult to reduce and the time to answer is far too long. Technologies involving the preparation of a test containing several antibodies, each specific to a target pathogen, suffer from the continuing evolution of pathogen antigenic binding sites, the difficulty of dehydrating proteins for transport such that they remain active, and the cost associated with the isolation and production of an antibody for each target pathogen. The present invention seeks to provide a method and apparatus for identifying a target polymer such as a nucleic acid which is an improvement over these prior art approaches.

WO 2009/030953 discloses a sequencing apparatus in which inter alia the sequence of nucleotides (bases or base pairs) in a single- or double-stranded polynucleotide sample (e.g. naturally occurring RNA or DNA) is read by translocating the same through a nano-perforated substrate provided with plasmonic nanostructures juxtaposed within or adjacent the outlet of the nanopores. In this device, the plasmonic nanostructures define detection windows (essentially an electromagnetic field) within which each nucleotide (optionally labelled) is in turn induced to fluoresce or Raman scatter photons in characteristic way by interaction with incident light. The photons so generated are then detected remotely, multiplexed and converted into a data stream whose information content is characteristic of the nucleotide sequence associated with the polynucleotide. This sequence can then be recovered from the data stream using computational algorithms embodied in corresponding software programmed into a microprocessor integral therewith or in a computing device attached thereto.

US 2005/0084912 discloses a method and apparatus for enhanced nano-spectroscopic scanning in which the sequence of successive nucleotides of a DNA analyte translocating through a nanopore is determined by Raman spectroscopy using a detector comprising a nanolens and a tip region provides with one or more plasmon resonance particles.

EP 2196796 discloses a single molecule optical spectroscopy method in which DNA is translocated through a nanoperforated membrane and the constituent nucleotide sequence thereof is determined by electrical means or by transmission optical spectroscopy which may be enhanced by plasmon resonance. EP 23243382 is directed to generally similar subject-matter where the force field created by the plasmons is used to influence the translocation speed of the DNA through the nanopore.

One difficulty that is encountered with devices such as those described above when used to detect the exact sequence of nucleotides in a DNA sample is reliably differentiating between adjacent nucleotide bases. For example where adjacent nucleotides in the nucleic acid strand are both labelled with fluorophores for detection by plasmon resonance enhanced fluorescence, the fluorophores tend to interfere with each other causing mutual quenching to a greater or lesser extent. This phenomenon is exacerbated when the translocation speed of the DNA molecule through the nanopore is high leading to the need for high detection rates. We have now found that this difficulty can be overcome by labelling higher order nucleotides structures in the chain as opposed to the individual nucleotides themselves. Such higher order structures are suitably chosen so that they are separated from one another so that problems of quenching may be reduced or even eliminated. Whilst the data obtained by such a method is no longer a complete sequence of nucleotide in the sample, a distribution profile of the labelled higher order structures throughout the sample can be obtained which itself can provide much useful information.

In a first aspect of the present invention there is provided a method for identifying a target polymer. Suitably, this method comprises translocating a target polymer having detectable elements through an analysing device comprising a nanopore and a detection window, wherein the analysing device is capable of plasmon resonance to produce a localised electromagnetic field which defines the detection window. In this method, the detectable elements are detected as they pass through the detection window to produce a distribution profile of the detectable elements along the target polymer. Thereafter the target polymer can be identified by comparing its distribution profile to a reference set of distribution profiles for known polymers.

The method of the present invention can in principle be used to identify any type of target polymer that is of a suitable size for translocation through the nanopore referred to above and that has a substantially linear structure comprised of repeating units of one or more, preferably two or more, monomer units: including for example a wide range of polyolefins, and condensation polymers. It is however primarily designed to analyse substantially un-branched, linear polymeric biomolecules such as nucleic acids or linear polypeptides or proteins derived therefrom from and is particularly suitable for the analysis of nucleic acids.

The term “nucleic acid” as used herein means a polymer of nucleotides. Nucleotides themselves are sometimes referred to as bases (in single stranded nucleic acid molecules) or as base pairs (in double stranded nucleic acid molecules). Nucleic acids suitable for use as target polymers in the present invention are typically the naturally-occurring nucleic acids DNA or RNA or synthetic versions thereof. However the method can also be applied if desired to analogues such as PNA (peptide nucleic acid), LNA (locked nucleic acid), UNA (unlocked nucleic acid), GNA (glycol nucleic acid) and TNA (threose nucleic acid). The nucleic acids themselves in turn suitably comprise a sequence of at least some of the following nucleotides: adenine (A), cytosine (C), guanine (G), thymine (T) and uracil (U) 4-acetylcytidine, 5-(carboxyhydroxylmethyl)uridine, 2-O-methylcytidine, 5-carboxymethylaminomethyl-2-thiouridine, 5-carboxymethylamino-methyluridine, dihydrouridine, 2-O-methylpseudouridine, 2-O-methylguanosine, inosine, N6-isopentyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, 5-methoxyuridine, 5-methoxycarbonylmethyl-2-thiouridine, 5-methoxycarbonylmethyluridine, 2-methylthio-N6-isopentenyladenosine, uridine-5-oxyacetic acid-methylester, uridine-5-oxyacetic acid, wybutoxosine, wybutosine, pseudouridine, queuosine, 2-thiocytidine, 5-methyl-2-thiouridine, 2-thiouridine, 4-thiouridine, 5-methyluridine, 2-O-methyl-5-methyluridine and 2-O-methyluridine.

Typically, the length of the nucleic acid sequence is expressed in terms of the number of nucleotides it contains. For example, the term “kilobase” (kb) means 1000 nucleotides whilst “megabase” (Mb) means 1,000,000 nucleotides. Whilst the nucleic acid can be of any length it is typically at least 10 bases (for single stranded nucleic acids) or base pairs (for double stranded nucleic acids) long, more typically at least 20, 30, 40, 50, 60, 70, 80, 90, 100, 500 or more bases/base pairs long or 1 kb, 2 kb, 5 kb, 10 kb, 20 kb, 50 kb, 100 kb, 250 kb, 500 kb or up to 1 Mb or more long. In particular, the method of the present invention is useful for rapidly identifying a long sequence length nucleic acid for example one characteristic of a bacterial, viral or eukaryotic gene. Accordingly, the method can be used advantageously to identify rapidly a suspected pathogen in biological samples such as blood, sputum, urine or foodstuff.

It is a feature of the method of the present invention that the target polymer is further comprised of detectable elements. These detectable elements can be any element within or attached to the target polymer that exhibits a detectable characteristic as the latter passes through the detection window of the analysing device. Typically, a plurality of the same or different detectable elements are employed. The exact number of the detectable elements within or attached to the target polymer will depend to a certain extent on the latter's characteristics, e.g. its length, the number of different monomer units it is comprised of etc., together with the degree of uniqueness required for its characterisation. However the number of said detectable elements should always be less than the total number of monomer units in target polymer or that part of the target polymer being analysed and be characteristic of at least two adjacent polymer units in the chain. Thus, when the target polymer is a nucleic acid such as DNA, it will have at least two detectable elements within or attached to it each of which is characteristic of a sequence of the four characteristic constituent nucleotides A, G, C and T. Suitably the detectable elements are such that they are able to generate, either directly or indirectly, a corresponding characteristic signal when caused to pass through the detection window. In a preferred embodiment of the invention, this characteristic signal is suitably generated by the emission of photons characteristic of the detectable element fluorescing or Raman scattering incident light within the detection window. In the case of Raman scattering the detectable element may form part of the molecular structure of the target polymer itself or may subsist in an element attached thereto which is identifiable using Raman spectroscopy.

In one aspect of the invention, the detectable elements are generated using labels designed to be attachable only to certain specific sequences of nucleotides in the nucleic acid, for example specific sequences of at least 2 suitably at least 2, 3, 4, 5 or 6 adjacent nucleotides (for example sequences hereinafter referred to as 2-mers, 3-mers, 4-mers, 5-mers, 6-mers etc.). In this embodiment, the labels preferably comprise a fluorophore or a sub-element capable of generating Raman scattering attached to an oligonucleotide probe for example, a nucleotide 6-mer such as TTGTTT, AATTTT, TCGCCG and TTGCGC. Such probes can then be attached to the complementary sequence in a single strand of the nucleic acid by hybridisation thereby labelling the latter in regions where the former is compatible. This leads to a plurality of detectable elements being attached to the nucleic acid at various points which manifests as a unique distribution profile which can be likened to a ‘barcode’ which can be read. At the same time the detectable element is designed so that when it is attached to the nucleic acid the individual detectable elements are separated by at least 10 nucleotides preferably at least 20 nucleotides on the nucleotide chain. In practice this means that the separation should be in the range 10 to 1000, preferably 20 to 100 nucleotides. This separation can typically be achieved by targeting nucleotide sequences which are found to occur in a wide variety of species yet are not highly repeated in a given nucleic acid. The choice of these probes can be aided via modelling in silico of the binding of these probes to a wide variety of organisms. The detectable elements can be attached over the whole or a portion of the target polymer thereby allowing a distribution profile to be generated which is a characteristic of the whole or at least a portion of it. Preferably, the number of such detectable elements within or attached to the nucleic acid being analysed is less than 1000 preferably less than 500.

Where the characteristic signal is generated by fluorescence, it is preferred that the detectable elements include a fluorophore i.e. a fluorescent label, tag or marker chemically or physically bound thereto. Suitable fluorophores include the following commonly used materials: Alexa dyes, cyanine dyes, Atto Tec dyes, and rhodamine dyes. Examples include: Atto 633 (ATTO-TEC GmbH), Atto 740 (ATTO-TEC GmbH), Rose Bengal, Alexa Fluor™ 750 C₅-maleimide (Invitrogen), Alexa Fluor™ 532 C₂-maleimide (Invitrogen) and Rhodamine Red C₂-maleimide and Rhodamine Green. These fluorophores can each be attached to the nucleotides using techniques known in the art.

Once obtained, the distribution profile characteristic of the target polymer can be compared against a reference set of pre-determined profiles characteristic of known polymers, for example the profiles characteristic of the DNA of a range of known pathogens. It is an advantage of the method of the present invention, especially when the target polymer is DNA, that, although the information content of the distribution profile is considerably less than that of a complete DNA sequence, it is nevertheless sufficiently unique for the target DNA to be identified from amongst the reference set. This makes it much quicker and cheaper to identify unknown pathogen samples than by complete de novo DNA sequencing. The target polymer can be identified by comparing its profile against the reference set using best fit statistical methods or visual inspection. Typically, the comparison is performed computationally and can be based on a set of logic decision rules, on a range of regression and classification methods (linear or not), on pattern matching and machine learning methods (such as neural networks, kernel methods or graphical models). For example, the comparison can be performed by a computer that has a database or reference set of distribution profiles for target polymers and a memory containing instructions which, when executed by the processor, compare the distribution profile of the target polymer to the reference set. In the case where no matching is found the target polymer can be identified by other means and the distribution profile added to the reference set for future reference.

The computer instructions may also be capable of identifying distribution profiles corresponding to targets unknown by the database. This can be used, for example, to help quickly detect new outbreaks cause by mutated or even new pathogens.

In the method of the invention the target polymer having detectable elements is suitably analysed by translocating it through an analysing device comprising a nanopore having a detection window defined by a localised electromagnetic field generated by plasmon resonance. It is the interaction between this electromagnetic field, the detectable elements and any incident electromagnetic radiation impinging on the detection window which generates an increased level of fluorescence or Raman scattering which can be easily detected and analysed. Further information concerning the characteristics of this analysing device can be found in WO 2009/030953 the contents of which are incorporated herein by reference. Briefly, the analysing device comprises a nano-perforated substrate separating sample providing and receiving chambers. The nano-perforated substrate may either be fabricated from an inorganic insulator or from organic or biological material. Preferably the nano-perforated substrate is an inorganic insulator such as a silicon carbide wafer. Typically, the nanopore is between 1 nm and 100 nm in diameter preferably 1 nm to 30 nm, 1 nm to 10 nm, 1 nm to 5 nm or 2 nm to 4 nm. The target polymer is suitably caused to translocate from the sample to the receiving chambers via the nanopore by electrophoresis. Passage through the nanopore ensures that the target polymer translocates in a coherent, linear fashion so that it emerges from the outlet thereof in a monomer unit by monomer unit fashion enabling the detectable elements to be detected in sequence.

The analysing device is provided with a detection window juxtaposed either within the nanopore or adjacent its outlet. Typically this detection window is defined by one or more metallic moieties fabricated from gold or silver capable of undergoing plasmon resonance under the influence of incident electromagnetic radiation from a coherent source such as a laser. This plasmonic resonance generates the strong localised electromagnetic field through which the target polymer passes. The exact geometry of these metallic moieties determines the geometry of the detection window and hence affects the nature of the interaction with the detectable elements. For example, the geometry of the detection window can be chosen so as to be optimised for increased photon emission, rather than for lateral localisation. This is achieved by producing detection windows with a greater z length (the dimension along which the polymer translocates), and modifying their geometry appropriately in the x and y dimensions in order to ensure their peak plasmonic resonance frequency is maintained at a desired wavelength. By differently dimensioning the detection window for the strength of signal rather than resolution, improvements can be made in detecting target polymers having detectable elements for which sub-nanometre localisation is not required. Preferably, the detection window is sized so that more than one detectable element can be in the detection window at the same time and is such that the length in the z dimension is from 1 to 100 preferably from 10 to 50 nanometres.

The signal generated by the interaction of the detectable elements and the electromagnetic field can be detected by a detector such as a photocounter in the case of fluorescence or a spectrometer in the case of Raman scattering. The output of such a device will typically be an electrical signal characteristic of the target polymer's distribution profile.

The method of the present invention may suitably employ multiple detectors and multiple analysing devices. For example, an array of pairs of detectors and analysing devices may be used with each detector being arranged to detect photons generated using its paired analysing device. Other detectors including other detectors for detecting fluorescence such as a photomultiplier or single photon avalanche diode may be used.

In a second aspect of the present invention there is provided an apparatus for identifying a target polymer the apparatus comprising: an analysing device comprising a nanopore having a detection window, wherein the analysing device is capable of plasmon resonance to produce a localised electromagnetic field which defines the detection window; a detector for detecting detectable elements of the target polymer as they pass through the detection window to produce a distribution profile of the detectable elements along the polymer; and a computer system for comparing the distribution profile to a reference set of distribution profiles for known polymers. The computer system typically comprises a memory and a processor. Computer executable instructions can be provided which when executed by the processor compare the distribution profile of the target polymer to a reference set of distribution profiles for target polymers to identify the target polymer.

The present invention will now be exemplified by the following figures in which:

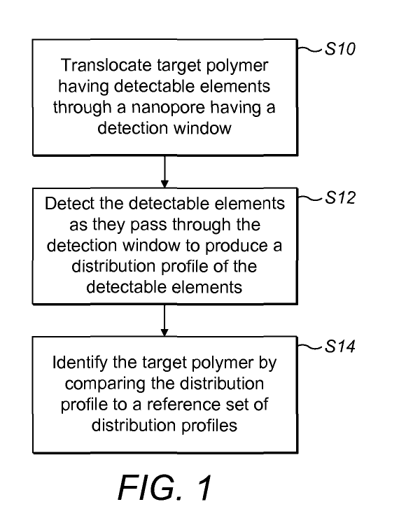

FIG. 1 is a flow diagram showing a method in accordance with an aspect of the present disclosure;

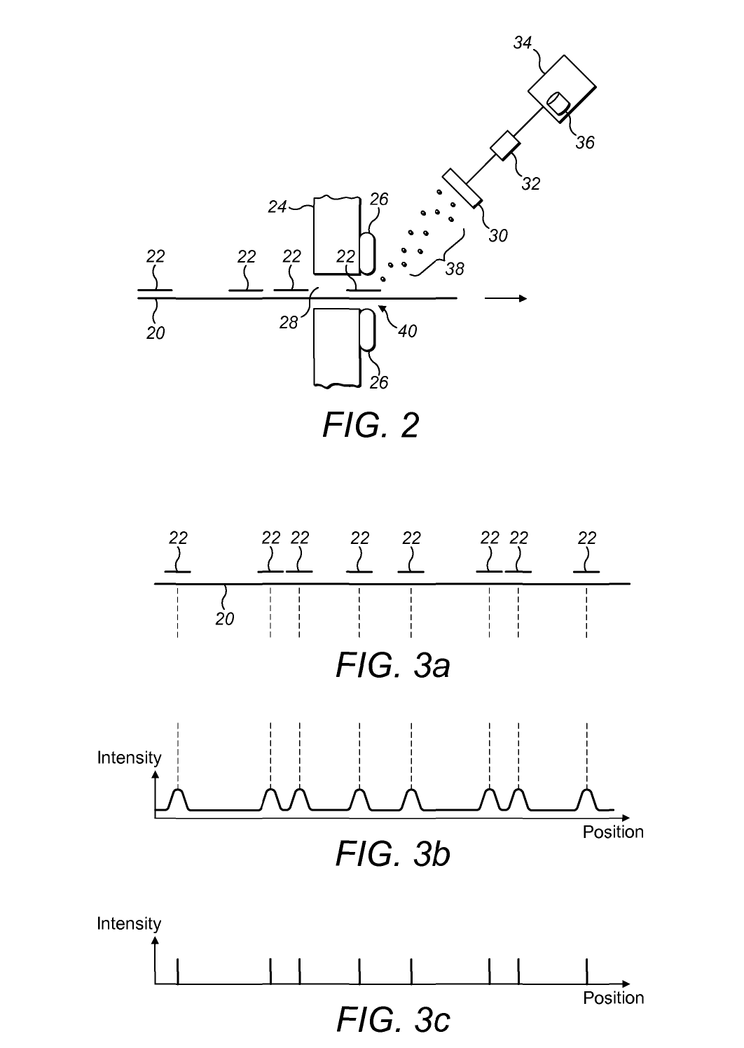

FIG. 2 schematically illustrates an apparatus for performing a first embodiment of the method of FIG. 1;

FIG. 3a illustrates the target polymer shown in FIG. 2;

FIG. 3b illustrates a distribution profile for the target polymer of FIG. 3a;

FIG. 3c illustrates an alternative representation of the distribution profile of FIG. 3b and

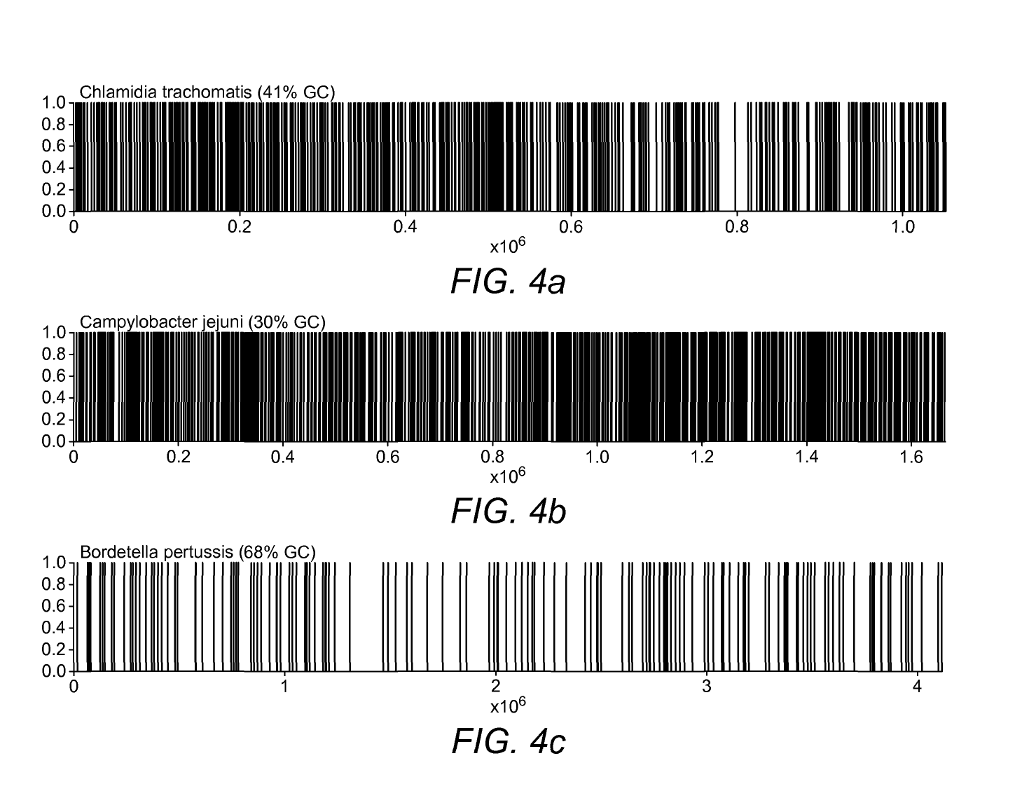

FIGS. 4a to 4c show some example distribution profiles.

FIG. 1 represents a flow diagram showing a method in accordance with the present invention. The method comprises, at step S10, translocating a target polymer having detectable elements (in this case fluorophores) through a nanopore having a detection window. The nanopore is part of an analysing device which has a gold plasmonic structure that is capable of plasmon resonance under incident laser light to produce a localised electromagnetic field which defines the detection window. At step S12, the detectable elements are detected as they pass through the detection window to produce a distribution profile characteristic of the fluorescence produced by the various fluorophores. At step S14, the target polymer is identified by comparing the distribution profile to a reference set of distribution profiles for known polymers.

FIG. 2 schematically illustrates an apparatus for performing the method of FIG. 1 comprising an analysing device 24, a photodetector 30, a data acquisition card 32 and a computer 34. 24 comprises a non-electrically conducting silicon carbide wafer perforated with a plurality of 4 nm diameter nanopores 28 and associated gold plasmonic structures 26 (doughnut shaped) juxtaposed over the outlet of 28 to define detection windows 40. In use, an unknown DNA sample of a pathogen 20 (isolated from a blood sample) is labelled with sequence-specific fluorescent markers 22 and caused to translocate though 28 and 40 by electrophoresis. 26 generate a localised electromagnetic field around the outlets of 28 which interacts with 22 causing them to fluoresce and emit photons 38 which are captured by 30. A laser (not shown) of frequency 750 nm and power 12 uW is used to induce the plasmon resonance. In one example, each of 22 comprises one of the four oligionucleotide 6-mers TTGTTT, AATTTT, TCGCCG and TTGCGC each of which are attached to different fluorophores and bound to the complementary sequences in 20 by hybridisation. Each of 22 are located at least 100 nucleotides apart from each other on 20. The total number of detectable elements so created is 400.

32 is used to receive the output of 30 and transfer it to 34 for analysis. The output is an electrical signal representing the distribution profile in the form of a profile of fluorescence over the length of at least a portion of 20. 34 comprises a processor and memory connected to a central bus structure which is in turn connected to a display via a display adapter and one or more input devices (such as a mouse and/or keyboard). 34 further comprises a communications adapter which is also connected to the central bus. The communications adapter can receive communications, in particular communications containing new distribution profiles for new polymers, which can be sent to the computer over a suitable communications link such as the internet.

The computer 34 has a database or reference set 36 of distribution profiles for known polymers and the memory of the computer contains instructions which when executed by the processor compare the measured distribution profile of the target polymer to this reference set to identify the target polymer using pattern matching software.

FIG. 3a illustrates 20 in the form of a nucleic acid and the detectable elements in the form of the fluorescent markers 22. FIG. 3b illustrates the distribution profile in the form of the intensity of the detected fluorescence over the length of at least a portion of 20. FIG. 3c shows an alternative representation of the distribution profile of FIG. 3b.

FIGS. 4a-4c use a similar representation to that of FIG. 3b and show the distribution profiles for Chlamidia trachomatis, Campylobacter jejuni and Bordetella pertussis, respectively.

1. A method for identifying a target polymer, comprising:
translocating a target polymer having detectable elements through an analysing device comprising a nanopore having a detection window, wherein the analysing device is capable of plasmon resonance to produce a localised electromagnetic field which defines the detection window;
detecting the detectable elements as they pass through the detection window to produce a distribution profile of the detectable elements along the target polymer; and
identifying the target polymer by comparing the distribution profile to a reference set of distribution profiles for known polymers.

2. A method according to claim 1 wherein the number of detectable elements is less than the number of the monomer units in the target polymer.

3. A method according to claim 2, wherein the wherein the target polymer is a nucleic acid and the detectable elements are oligonucleotides complimentary to at least two adjacent nucleotides therein.

4. A method according to claim 3, the detectable elements are oligonucleotides complimentary to at least four adjacent nucleotides in the nucleic acid.

5. A method according to claim 4, wherein the detectable elements are oligonucleotides complimentary to at least six adjacent nucleotides in the nucleic acid.

6. A method according to claim 1, wherein the detectable element includes a fluorophore.

7. A method according to claim 1, wherein the detectable element is identifiable using Raman spectroscopy.

8. A method according to claim 1, wherein the distribution profile is a profile of fluorescence over the length of at least a portion of the target polymer and wherein the plasmon resonance enhances fluorescent properties of the detectable elements.

9. A method according to claim 7, wherein the distribution profile is a profile of Raman scattering over the length of at least a portion of the target polymer and wherein the plasmon resonance increases the level of Raman scattering from the detectable elements.

10. A method according to claim 1, wherein the nanopore located in a nano-perforated substrate which is an inorganic insulator.

11. A method according to claim 1, wherein the detection window is sized so that more than one detectable element can be in the detection window at the same time.

12. A method according to claim 1, wherein the detection window is between 1 nanometre and 100 nanometres

13. A method according to claim 1, wherein the detection window is between 10 and 50 nanometres.

14. A method according to claim 2, wherein the detectable element includes a fluorophore.

15. A method according to claim 3, wherein the detectable element includes a fluorophore.

16. A method according to claim 4, wherein the detectable element includes a fluorophore.

17. A method according to claim 5, wherein the detectable element includes a fluorophore.

18. A method according to claim 2, wherein the detectable element is identifiable using Raman spectroscopy.

19. A method according to claim 3, wherein the detectable element is identifiable using Raman spectroscopy.

20. A method according to claim 4, wherein the detectable element is identifiable using Raman spectroscopy.
